(19) United States
(12) Patent Application Publication
(10) Pub. No.: US 2009/0263281 A1
(43) Pub. Date: Oct. 22, 2009

(54) AUTOMATIC ANALYZER

(75) Inventors: Emiko Ushiku, Hitachinaka (JP); Tomonori Mimura, Kasama (JP)

Correspondence Address: MATTINGLY & MALUR, P.C., 1800 DIAGONAL ROAD, SUITE 370, ALEXANDRIA, VA, 22314, US

(73) Assignee: HITACHI HIGH-TECHNOLOGIES CORPORATION, Tokyo (JP)

(21) Appl. No.: 12/423,337

(22) Filed: Apr. 14, 2009

(30) Foreign Application Priority Data
Apr. 17, 2008 (JP) 2008-107389

(51) Int. Cl. G01N 33/00

(52) U.S. Cl. 422/68.1

(57) ABSTRACT

An automatic analyzer is capable of recording contents of a sample subjected to a special treatment to take for an unusual condition to ensure traceability of the sample. The automatic analyzer has functions to easily perform a process for a series of operations including the special treatment, a request for a re-measurement after the special treatment, and confirmation of the result of the re-measurement. The automatic analyzer has a database dedicated for a sample accompanied by an unusual condition and records contents of the special treatment in the database. The automatic analyzer has a dedicated re-measurement request screen, a dedicated measurement result screen and a dedicated special treatment trace screen. The automatic analyzer has a function of guiding a method for coping with each unusual condition.

### BACKGROUND OF THE INVENTION

1. Field of the Invention

The present invention relates to an automatic analyzer for qualitatively and quantitatively analyzing a biological sample such as blood and urine. The invention more particularly relates to an automatic analyzer and an automatic analysis system, each of which has functions to easily perform a process for a series of operations including a special treatment to take for an unusual condition, a request for a re-measurement after the special treatment, and confirmation of the result of the re-measurement when an unusual condition attributable to the states of a sample or a reagent during analysis occurs.

2. Description of the Related Art

A typical automatic analyzer receives a constant amount of a sample and a constant amount of a reagent, causes the sample and the reagent to react in a reaction vessel and measures absorbance or fluorescence. The automatic analyzer analyzes samples of patients in accordance with multiple analysis items and therefore uses multiple reagents to treat the samples. When samples of patients and reagents are set to the automatic analyzer and measurements are completed without any trouble during analyses, there is no special problem. However, the following unusual conditions may occur during the analyses since the analyses are clinical examinations.

### 1. Sample

A taint, scratch or the like of a barcode, and a failure to read the barcode for the reason that the barcode is unclearly printed
Detection of clogging of a sample probe due to a foreign material such as fibrin
A lack of sample

### 2. Reagent

A lack of reagent
A new reagent for which calibration result is not known

These unusual conditions are not attributed to the automatic analyzer and are unavoidable as long as a routine measurement is performed in a clinical examination. Operations that are to be performed after occurrence of any of the unusual conditions are different from a normal routine operation.

For example, it is assumed that analyses are requested to be performed in accordance with fifteen analysis items for a certain sample and that clogging of a sample probe is detected during the analysis for the fifth item. In that case, the analyses for the subsequent analysis items for which measurement is to be performed will be interrupted. An operator extracts the sample from the automatic analyzer and removes a foreign material such as fibrin. Even if the operator wants to immediately resume the analysis for any of such analysis items, the analysis cannot be performed again using the same IDs since sample IDs will be duplicated during a period of time until results of the first four items for which sampling has been completed are output. Thus, the operator operates the automatic analyzer for the unusual condition, e.g., sets the sample from a general sample to an urgent sample or manually enters a virtual ID into the automatic analyzer. After that, the operator operates the automatic analyzer to cause the automatic analyzer to perform a re-measurement on the sample. The result of the re-measurement is reported without being distinguished from the result of a measurement performed in the normal routine operation.

JP-A-8-262031 discloses an automatic analyzer that displays an identification of the occurrence of an unusual condition during such an analysis in accordance with the degree of emergency.

The contents of a treatment to be performed for an unusual condition are complicated as described above and are entrusted to the operator. Thus, the operator may erroneously enter data and perform erroneous operation. Even when such an error is found, the conventional technique cannot trace why and where the error took place.

In addition, it is a cumbersome task to search samples one by one for finding a sample subjected to a special treatment to take for an unusual condition and confirm measurement results of the samples.

### SUMMARY OF THE INVENTION

When an unusual condition attributable to the states of a sample or a reagent occurs, the operator understands the status of the automatic analyzer, identifies and deals with the cause of the unusual condition, and performs a series of tasks for re-measurement. In the course of the process, the automatic analyzer may be stopped and restarted whereas only the sample or a part of items is problematic and the automatic analyzer may not need to be stopped. In order to treat only a specific sample, the operator extracts the sample from the automatic analyzer and performs troubleshooting in order to introduce a special treatment to take for an unusual condition into a normal operation (normal treatment to be performed by the automatic analyzer) of the automatic analyzer. A typical introduction method used is to measure the sample as an urgent sample.

The special treatment for such an unusual condition is different from a routine operation and complicated. Thus, the operator may erroneously enter data and erroneously operate the automatic analyzer. Even when such an error is found, the conventional technique cannot trace why and where the error took place. In addition, the result obtained through the special treatment is reported without being distinguished from the result of a measurement performed in the normal routine operation. Thus, it is a cumbersome task to search samples one by one for finding a sample subjected to a special treatment for such an unusual condition and confirm measurement results of the samples.

An object of the present invention is to provide an automatic analyzer and automatic analysis system, each of which is capable of being rapidly handled when an unusual condition occurs and ensuring traceability during a treatment for the unusual condition.

When a lack of reagent, clogging of a sample probe, a failure in reading a barcode, a lack of sample, a calibration error, or a quality control error occurs or the reagent is expired during a measurement performed for a certain analysis item, an analysis for the analysis item is regarded as a failure. In order to perform the analysis for the analysis item, it is necessary to request a re-analysis with a measurement number (sequence number) changed and with a sample ID and a patient ID unchanged or to request a re-analysis with a sample ID and a patient ID changed as another sample. When the result of the sample thus measured is displayed on the basis of sample IDs and patient IDs, the analysis result is displayed as if the result of the sample was that of another sample. Thus, the re-analysis result cannot be determined to be either the result of a re-measurement performed due to an error or the initial measurement result. To avoid this problem, the automatic analyzer according to the present invention includes an analyzer for analyzing a sample in accordance with a first analysis item and in response to a first request; and a storage mechanism for associating, with identification information used to identify a sample subjected to a special treatment, measurement data on the sample analyzed by the analyzer in accordance with the first analysis item and in response to a second request different from the first request, and storing the measurement data associated with the identification information.

The operator does not need to stop the automatic analyzer during a process for a series of operations to understand the status of the automatic analyzer, identify and deal with the cause of the unusual condition and perform a re-measurement. In order to treat only a sample accompanied by an unusual condition, the operator handles the sample and operates the automatic analyzer in order to introduce a special treatment to take for an unusual condition into a normal operation (normal treatment to be performed by the automatic analyzer) of the automatic analyzer. A process that is performed on the sample subjected to the special treatment can be clarified separately from that performed on the sample subjected to the normal treatment.

In order to clarify the process that is performed on the sample subjected to the special treatment, the following functions are required.

1. The automatic analyzer has the following screens required when an unusual condition occurs.

(1) Dedicated special treatment trace screen that displays contents of a process for an unusual condition

(2) Dedicated re-measurement request screen that is used to restart an analysis after an occurrence of an unusual condition

(3) Dedicated measurement result screen that displays the result of a measurement of a sample subjected to a special treatment to take for an unusual condition and is used to confirm and approve the measurement result

2. A sample subjected to a special treatment to take for an unusual condition is measured through troubleshooting by an operator. Thus, a process that is to be performed before the measurement is different from a process that is to be performed on a normal sample. It is necessary that the measurement result of the sample subjected to the special treatment be confirmed and approved. Thus, an entrance and exit for the sample subjected to the special treatment are different from those for a sample subjected to the normal treatment. The entrance means an entrance of the data base, while the exit means an exit of the data base. The entrance and the exit vary depending on whether or not the sample is subjected to a special treatment to take for an unusual condition. The automatic analyzer has a database dedicated for a sample subjected to a special treatment for an unusual condition to manage a measurement process.

3. The automatic analyzer records contents of an unusual condition, a sample number after a special treatment for an unusual condition, a sample ID, a setting position, a measurement item, a measurement result and the like in the database dedicated for a sample accompanied by an unusual condition in order to ensure traceability.

4. The automatic analyzer has a function of guiding a process for each unusual condition in order to clarify a method for coping with the unusual condition.

5. The automatic analyzer has a function of adding a mark to the measurement result based on the type of an unusual condition in order to identify the measurement result.

The operator copes with an unusual condition in accordance with a guidance displayed for each cause of the unusual conditions on the re-measurement request screen to reduce the risk of an error in operation. The measurement result of a sample subjected to the special treatment for the unusual condition is stored in a database different from a database storing measurement results of normally treated samples and displayed on the dedicated measurement result screen, which improves operability of the automatic analyzer. In addition, a process for confirmation and approval by an administrator is performed. Thus, only the measurement result of a sample subjected to a special treatment to take for an unusual condition, which is confirmed and approved by the administrator, can be reported to an external computer. Furthermore, the automatic analyzer ensures traceability by recording the contents of the special treatment.

### BRIEF DESCRIPTION OF THE DRAWINGS

FIG. 1 is a block diagram showing an automatic analyzer according to an embodiment of the present invention.

FIG. 2 is a work flow of the automatic analyzer according to the embodiment of the present invention.

FIG. 3 is a diagram showing a measurement result screen that is dedicated for a sample subjected to a special treatment to take for an unusual condition and is included in the automatic analyzer according to the embodiment of the present invention.

FIG. 4 is a diagram showing a re-measurement request screen that is dedicated for a sample subjected to a special treatment to take for an unusual condition and is included in the automatic analyzer according to the embodiment of the present invention.

FIG. 5 is a diagram showing a special treatment trace screen that is dedicated for a sample subjected to a special treatment to take for an unusual condition and is included in the automatic analyzer according to the embodiment of the present invention.

FIG. 6 is a flow of a process performed by the automatic analyzer according to the embodiment of the present invention when a failure to read a barcode for a sample occurs.

FIG. 7 is a flow of a process performed by the automatic analyzer according to the embodiment of the present invention when a sample is insufficient.

FIG. 8 is a flow of a process performed by the automatic analyzer according to the embodiment of the present invention when a sample probe is clogged and a foreign material is successfully removed from the sample probe.

FIG. 9 is a flow of a process performed by the automatic analyzer according to the embodiment of the present invention when a sample probe is clogged and removal of a foreign material from the sample probe fails.

FIG. 10 is a flow of a process performed by the automatic analyzer according to the embodiment of the present invention when a reagent is insufficient.

### DESCRIPTION OF THE PREFERRED EMBODIMENTS

An embodiment of the present invention is described below with reference to the accompanying drawings.

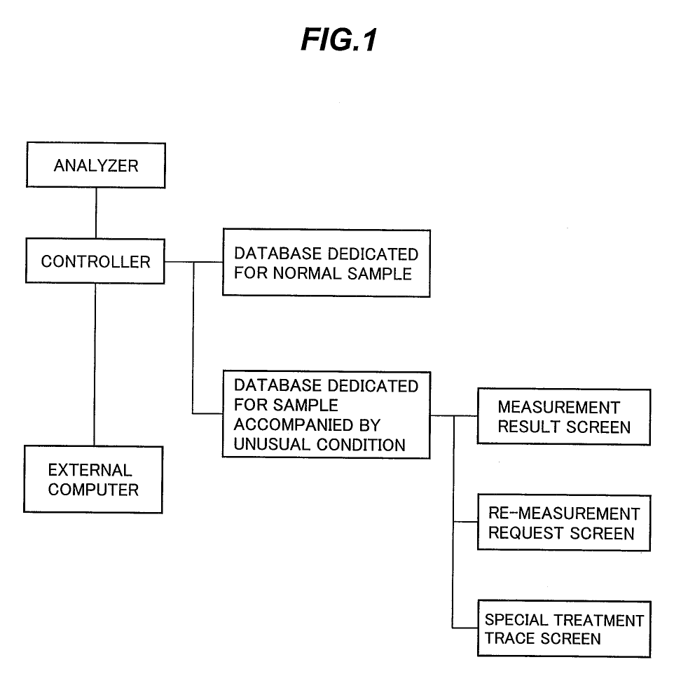

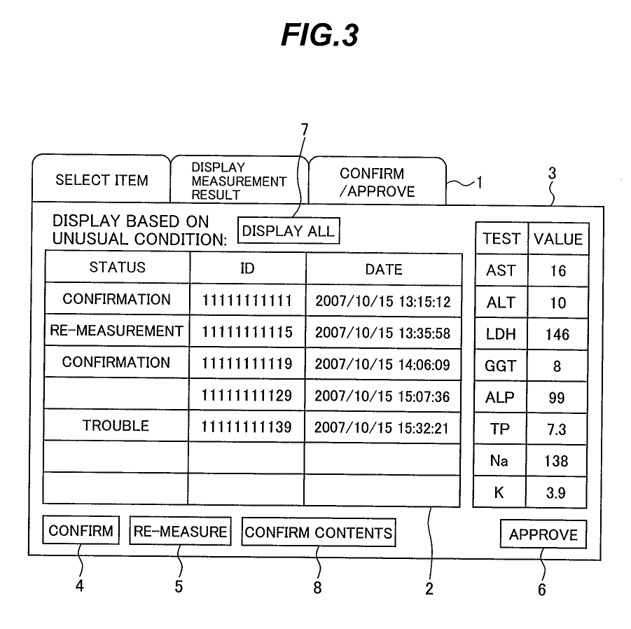

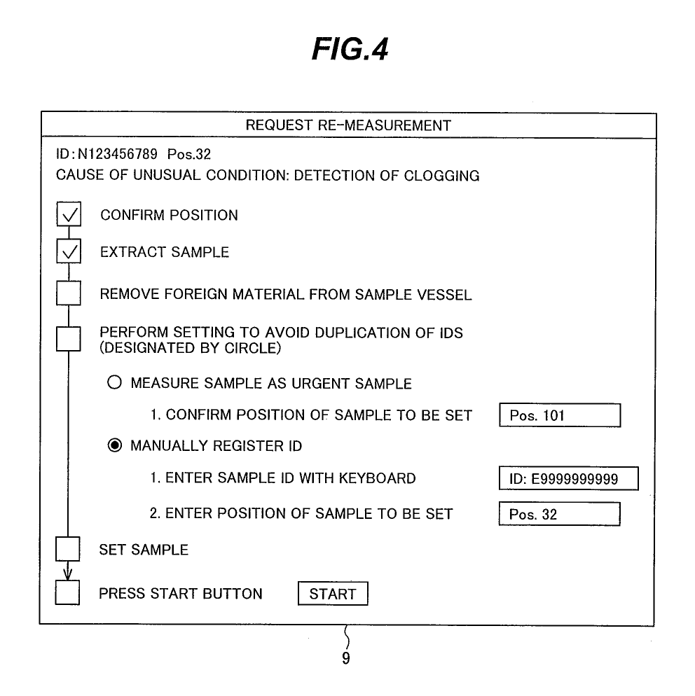

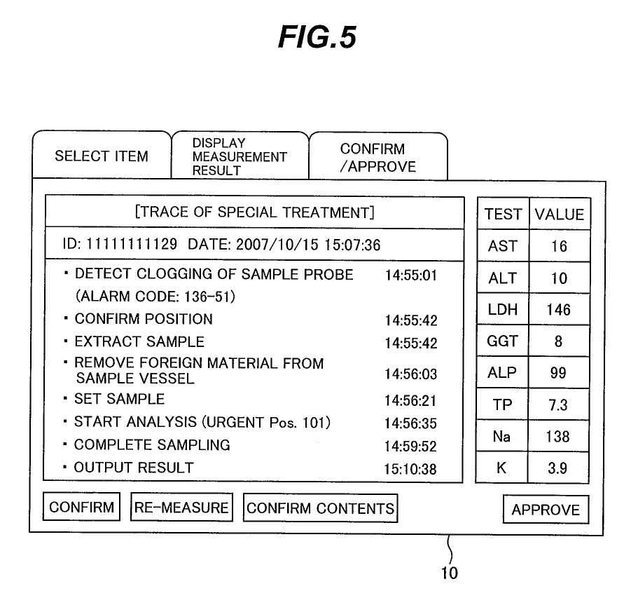

An automatic analyzer according to the embodiment of the present invention has a data processor. The data processor is described below with reference to FIG. 1. The automatic analyzer according to the embodiment includes an analyzer and a controller (storage mechanism). The controller is connected with an external computer. The controller is adapted to transmit and receive measurement request information and a measurement result to and from the external computer. The controller has a database dedicated for a normal sample and a database dedicated for a sample accompanied by an unusual condition. The database dedicated for a normal sample is used to manage a sample normally treated. The database dedicated for a sample accompanied by an unusual condition is used to manage a sample subjected to a special treatment for an unusual condition. The database dedicated for a sample accompanied by an unusual condition includes data on the following three screens: a measurement result screen; a re-measurement request screen; and a special treatment trace screen. The three screens are used only for samples subjected to a special treatment performed for unusual conditions. The measurement result screen is denoted by reference numeral 1 shown in FIG. 3. The re-measurement request screen is denoted by reference numeral 9 shown in FIG. 4. The special treatment trace screen is denoted by reference numeral 10 shown in FIG. 5.

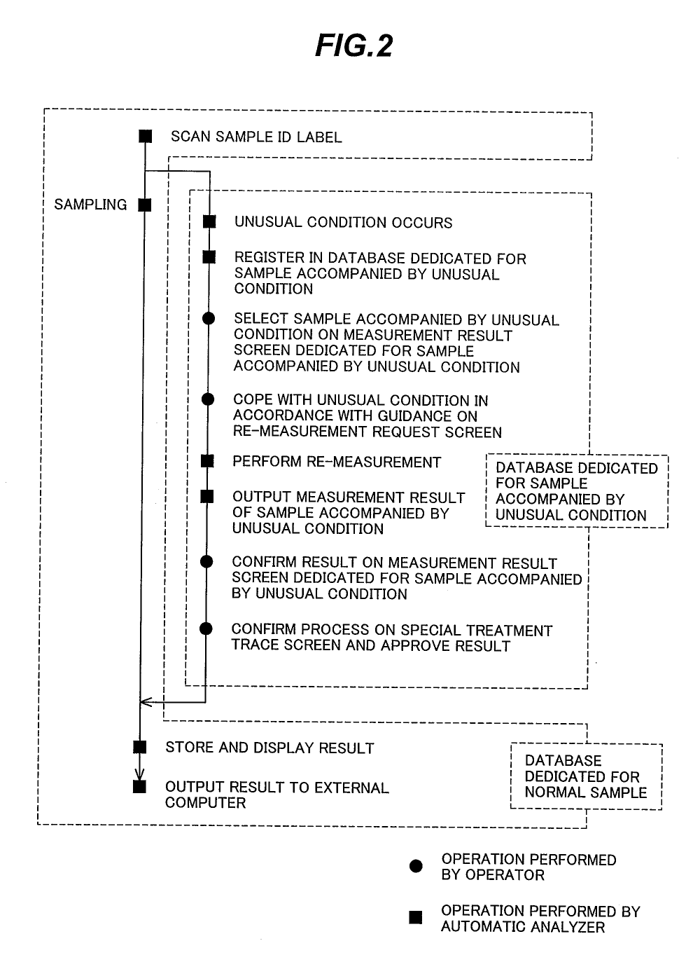

The outline of a work flow of the automatic analyzer is described with reference to FIG. 2. An operator sets samples to the automatic analyzer and presses a start button of the automatic analyzer. Then, the automatic analyzer automatically scans an identification (ID) label of the sample and samples the sample. The automatic analyzer then outputs the results of the scanning and the sampling. The output results are stored in the database dedicated for a normal sample, displayed on a screen and output to a printer and the external computer. These operations are collectively regarded as a routine operation.

In contrast, when an unusual condition occurs (for example, when a reagent is not sufficient, or the sample is stuck, or a barcode is not correctly read), the operator understands the status of the automatic analyzer, identifies and deals with the cause of the unusual condition, and performs a series of handling for re-measurement. In the case where the sample accompanied by the unusual condition is treated without a suspension of the automatic analyzer in this process, the operator handles the sample and operates the automatic analyzer in order to introduce a special treatment to take for an unusual condition into a normal operation (normal treatment that is to be performed by the automatic analyzer) of the automatic analyzer.

The sample in question is registered in the database dedicated for a sample subjected to a special treatment and displayed on the dedicated measurement result screen 1. The operator selects the corresponding sample on the measurement result screen 1 and opens the dedicated re-measurement request screen 9. After that, the operator instructs the automatic analyzer to perform a re-measurement in accordance with a guidance displayed on the re-measurement request screen 9. The result of the re-measurement is displayed on the measurement result screen 1. The operator confirms the result of the re-measurement on the measurement result screen 1. In addition, an administrator approves the result of the re-measurement. In this case, the administrator can confirm the contents of the special treatment in chronological order by opening the special treatment trace screen 10. Therefore, the sample subjected to a special treatment can be handled as a normal sample through the confirmation and the approval. The result of the sample subjected to a special treatment handled as the normal sample is transferred into the database dedicated for a normal sample and output to the external computer.

Next, the measurement result screen 1, the re-measurement request screen 9 and the special treatment trace screen 10, which are dedicated for a sample accompanied by an unusual condition, are described below.

FIG. 3 shows the measurement result screen 1 dedicated for a sample accompanied by an unusual condition. The measurement result screen 1 has a status field. A sample that cannot be completely measured due to an occurrence of an alarm (generated due to, for example, a lack of reagent, a clogged sample probe, or a failure to read a barcode) is registered in the measurement result screen 1. The status field for the sample that cannot be completely measured indicates “trouble”. In order to re-measure the sample in question, a re-measurement button 5 is pressed to open the re-measurement request screen 9 shown in FIG. 4. The re-measurement request screen 9 displays an ID attached to a sample accompanied by an unusual condition, the position of the sample and the cause of the unusual condition. The re-measurement request screen 9 shows procedures required to be performed by the operator before the re-measurement. The procedures are displayed for each of causes of unusual conditions in a flow format. The operator operates the automatic analyzer in accordance with a guidance displayed on the re-measurement request screen 9 to cause the analyzer to perform each operation. When the operator presses a checkbox, the time when an operation indicated by the checkbox is performed is recorded in the automatic analyzer. When the operator finally presses a start button, the re-measurement is performed. The result of the re-measurement is displayed on the measurement result screen 1 shown in FIG. 3. The operator confirms a measured value for each of samples subjected to a special treatment. When there is no problem with the measured values, the operator presses a confirmation button 4. The administrator approves the confirmed results. Then, the confirmed results are transferred into the database dedicated for a normal sample and output to the external computer in real time or collectively. A process for entering a user ID and pass word of a person who confirms the measured results or entering a user ID and pass word of a person who approves the measured results may be performed to confirm and approve each sample. When the operator presses a content confirmation button 8 in order to confirm and approve the measured results, the special treatment trace screen 10 shown in FIG. 5 is displayed. Contents of the special treatment are displayed in chronological order on the special treatment trace screen 10.

Detail examples are described below for each of causes of unusual conditions.

### (1) In the Case Where an Error in Reading a Barcode Indicative of a Sample ID Occurs

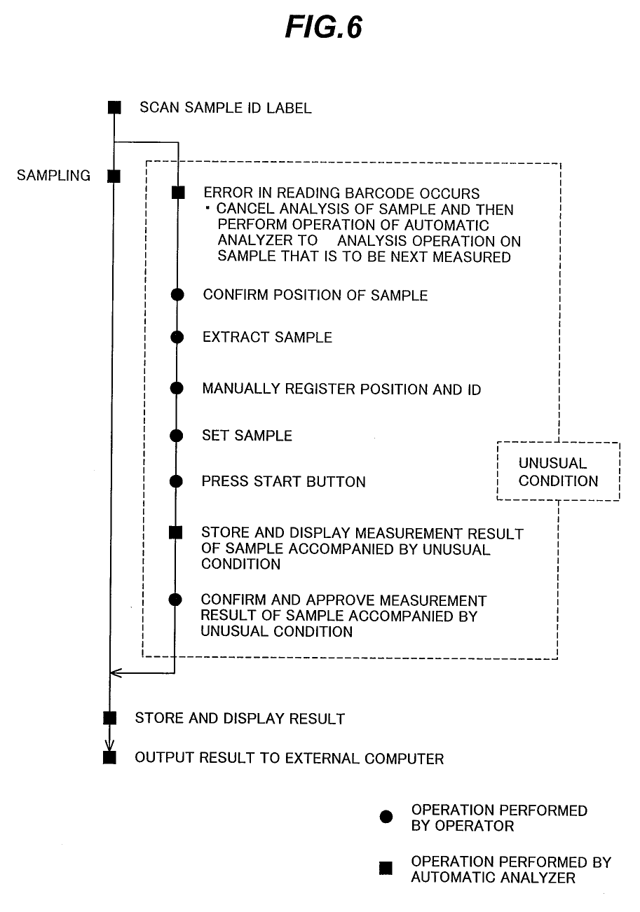

FIG. 6 is a flow of a process that is to be performed in the case (1). An ID label of a sample set in a disk or rack is scanned by a barcode reader. When the barcode reader cannot read the sample ID label due to a taint, scratch or the like of the label, the automatic analyzer generates an alarm indicative of an error in reading the barcode for the sample and cancels an analysis of the sample. In this case, only the sample for which the alarm is generated is required to be treated. Thus, the automatic analyzer does not need to be stopped. The operator handles the sample and operates the automatic analyzer to introduce a special treatment to take for an unusual condition (referred to as special treatment) into the normal operation of the automatic analyzer.

The sample treated in the case (1) is registered in the measurement result screen 1 dedicated for a sample accompanied by an unusual condition. The status field for the sample treated in the case (1) indicates “trouble”. The operator selects the sample treated in the case (1) on the measurement result screen 1 and presses the re-measurement button 5. Then, the re-measurement request screen 9 is displayed. The re-measurement request screen 9 shows items (shown in FIG. 6) of operations in a flow format. The items of the operations are to be performed by the operator or to be performed by the automatic analyzer. The operator extracts the sample in accordance with the guidance, and performs troubleshooting (manual registration of the position and ID of the sample and the like). The sample subjected to the troubleshooting is reset to the automatic analyzer and measured by the automatic analyzer.

The results of the measurement are output and stored in the database for a sample accompanied by an unusual condition. This database dedicated is different from the database that is dedicated for a normal sample and designed to store a sample normally treated. The results are displayed on the measurement result screen 1. The operator selects a target sample and confirms a measured value of the target sample for each item. After that, when the operator presses the confirmation button 4, the status field for the sample indicates “confirmation”. When the sample is required to be re-measured, the operator needs to press the re-measurement button 5. When the operator confirms samples subjected to all operations of the special treatment and then presses an approval button 6, data on the samples are erased from the screen 1, transferred into the database dedicated for a normal sample and output to the external computer in real time or collectively. The automatic analyzer records therein operations of the special treatment (shown in FIG. 6) and times when the operations are performed. In this case, the automatic analyzer records therein the operations and the times by using, as a trigger, the generation of the alarm indicative of the error in reading the barcode. Thus, the automatic analyzer can trace contents of the special treatment.

In the flow shown in FIG. 6, the result of the measurement of the sample subjected to the special treatment is displayed on the dedicated measurement result screen 1. The operator does not need to search a sample subjected to the auxiliary treatment from a lot of samples. Thus, the search performance can be improved. Since the processes for confirmation and approval by the administrator are performed, only approved measured results can be output to the external computer among measured results of samples subjected to the special treatment. By displaying the recorded operations of the special treatment and the recorded times on the special treatment trace screen 10 in order to confirm and approve the measured results, contents of the special treatment can be confirmed in chronological order on the special treatment trace screen 10.

### (2) In the Case Where an Alarm Indicative of a Lack of Sample is Generated

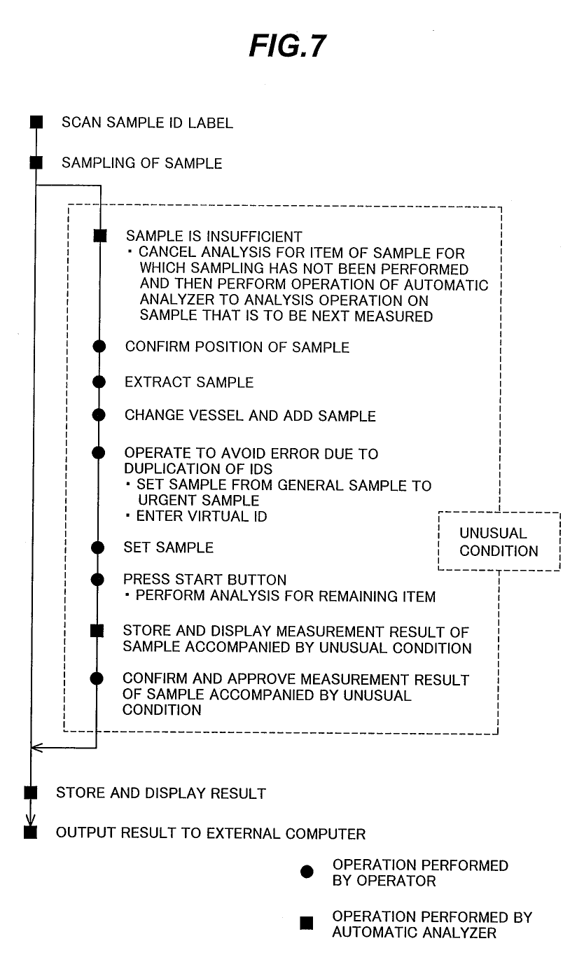

FIG. 7 shows a flow of a process that is to be performed in the case (2). When a remaining amount of a sample is reduced during sampling of the sample, the automatic analyzer adds an alarm indicative of a lack of the sample to an item of the sample for which sampling has not been performed. In this case, an analysis for an item indicative of already-performed sampling continues to be performed. In contrast, an analysis for a remaining item whose sampling has not been performed is cancelled. The automatic analyzer then performs an analysis operation on a sample that is to be next measured. In this case, only the sample for which the alarm indicative of a lack of sample is generated is required to be treated. Thus, the automatic analyzer does not need to stop. The operator handles the sample and operates the automatic analyzer to introduce a special treatment into the normal operation of the automatic analyzer.

The sample treated in the case (2) is registered in the measurement result screen 1. The status field for the sample treated in the case (2) indicates “trouble”. The operator selects the sample treated in the case (2) on the measurement result screen 1 and presses the re-measurement button 5. Then, the re-measurement request screen 9 is displayed. The re-measurement request screen 9 shows items (shown in FIG. 7) of operations in a flow format. The items of operations are to be performed by the operator or to be performed by the automatic analyzer. The operator extracts the sample from the automatic analyzer in accordance with the guidance. After that, the operator replaces a sample vessel with another sample vessel and adds a sample to the replaced sample vessel. Even if the operator wants to immediately resume the analysis for any of such analysis items, the analysis cannot be performed again using the same IDs since sample IDs will be duplicated during a period of time until results of the items for which sampling has been completed are output. A re-measurement cannot be performed using the same IDs. Therefore, the operator sets the sample from a general sample to an urgent sample or manually enters a virtual ID or the like on the dedicated re-measurement request screen 9 to avoid the duplication of the IDs. After that, the re-measurement is performed.

The results of the re-measurement are output and stored in the database dedicated for a sample accompanied by an unusual condition. This database is different from the database that is dedicated for a normal sample and designed to store a sample normally treated. The output results are displayed on the measurement result screen 1. The results of the measurement of the sample, which are confirmed and approved by the administrator, are transferred into the database dedicated for a normal sample and output to the external computer in real time or collectively. The automatic analyzer records unusual conditions shown in FIG. 7 such as contents of the alarm and screen operations performed to avoid the duplication of the IDs by using, as a trigger, the generation of the alarm indicative of the lack of the sample. Then, the recorded unusual conditions are displayed on the special treatment trace screen 10.

In the flow shown in FIG. 7, the performance for searching a sample subjected to the special treatment is improved in a similar way to the aforementioned example. In addition, reliability of the automatic analyzer is improved due to the processes for the confirmation and approval. Furthermore, traceability of the automatic analyzer is ensured by recording the contents of the special treatment for the unusual condition.

### (3) In the Case Where an Alarm Indicative of Detection of Clogging of a Sample Probe is Generated and a Foreign Material is Removed From the Sample Probe by a Cleaning Operation

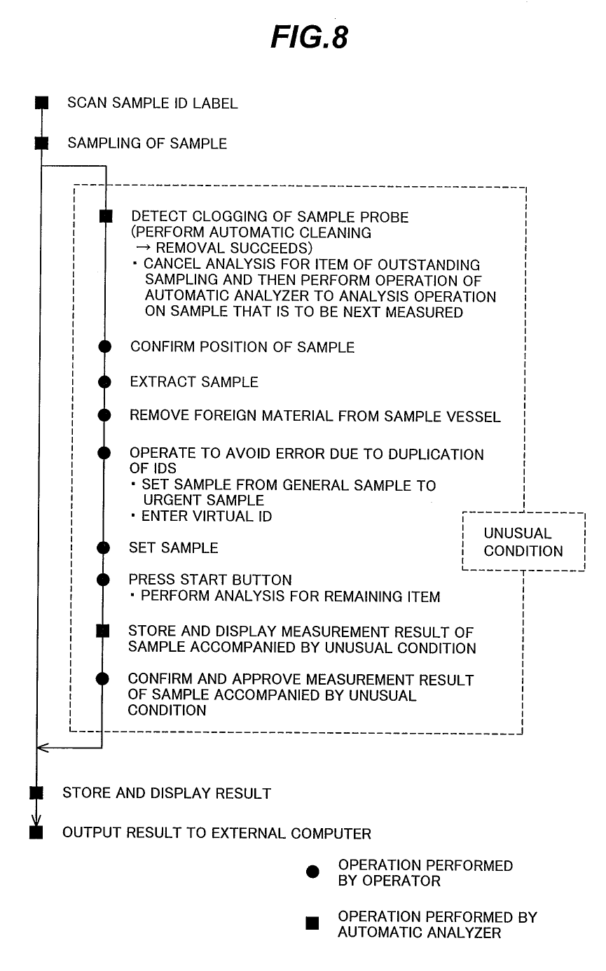

FIG. 8 shows a flow of a process that is to be performed in the case (3). When the sample probe is clogged due to a foreign material such as fibrin during sampling of a sample, the automatic analyzer adds an alarm indicative of detection of the clogging to an item of the sample for which sampling has not been performed. In this case, an analysis for an item for a sampled sample continues to be performed. In contrast, an analysis for a remaining item whose sampling has not been performed is cancelled, and the process proceeds to the cleaning operation. When the foreign material can be removed from the sample probe by the automatic cleaning operation, the automatic analyzer then performs an analysis operation on a sample that is to be next measured. In this case, only the sample for which the alarm indicative of detection of clogging of the sample probe is generated is required to be treated. Thus, the automatic analyzer does not need to stop. The operator handles the sample and operates the automatic analyzer to introduce a special treatment into the normal operation of the automatic analyzer.

The sample treated in the case (3) is registered in the measurement result screen 1, and the status field for the sample treated in the case (3) indicates “trouble”. The operator selects the sample treated in the case (3) on the measurement result screen 1 and presses the re-measurement button 5. Then, the re-measurement request screen 9 is displayed. The re-measurement request screen 9 shows items (shown in FIG. 8) of operations in a flow format. The items of operations are to be performed by the operator or to be performed by the automatic analyzer. The operator extracts the sample in accordance with the guidance, and removes the foreign material (such as fibrin) from a sample vessel. Even if the operator wants to immediately resume the analysis for any of such analysis items, the analysis cannot be performed again using the same IDs since sample IDs will be duplicated during a period of time until results of the items for which sampling has been completed are output. A re-measurement cannot be performed using the same IDs. Therefore, the operator sets the sample from a general sample to an urgent sample or manually enters a virtual ID or the like on the dedicated re-measurement request screen 9 to avoid the duplication of the IDs. After that, the re-measurement is performed.

The results of the re-measurement are output and stored in the database dedicated for a sample accompanied by an unusual condition. This database is different from the database that is dedicated for a normal sample and designed to store a sample normally treated. The output results are displayed on the measurement result screen 1. The results of the measurement of the sample, which are confirmed and approved by the administrator, are transferred into the database dedicated for a normal sample and output to the external computer in real time or collectively. The automatic analyzer records therein unusual conditions shown in FIG. 8 such as contents of the alarm and screen operations performed to avoid the duplication of the IDs by using, as a trigger, the generation of the alarm indicative of detection of clogging of a sample probe. Then, the recorded unusual conditions are displayed on the special treatment trace screen 10. Data on a sample that is to be measured next to the sample detected to be clogged can be preset to be automatically stored in the database dedicated for a sample subjected to a special treatment.

In the flow shown in FIG. 8, the performance for searching a sample subjected to the special treatment is improved in a similar way to the aforementioned examples. In addition, reliability of the automatic analyzer is improved due to the processes for the confirmation and approval. Furthermore, traceability of the automatic analyzer is ensured by recording the contents of the special treatment for the unusual condition.

### (4) In the Case Where the Alarm Indicative of Detection of Clogging of a Sample Probe is Generated and a Foreign Material Cannot be Removed From the Sample Probe by the Cleaning Operation

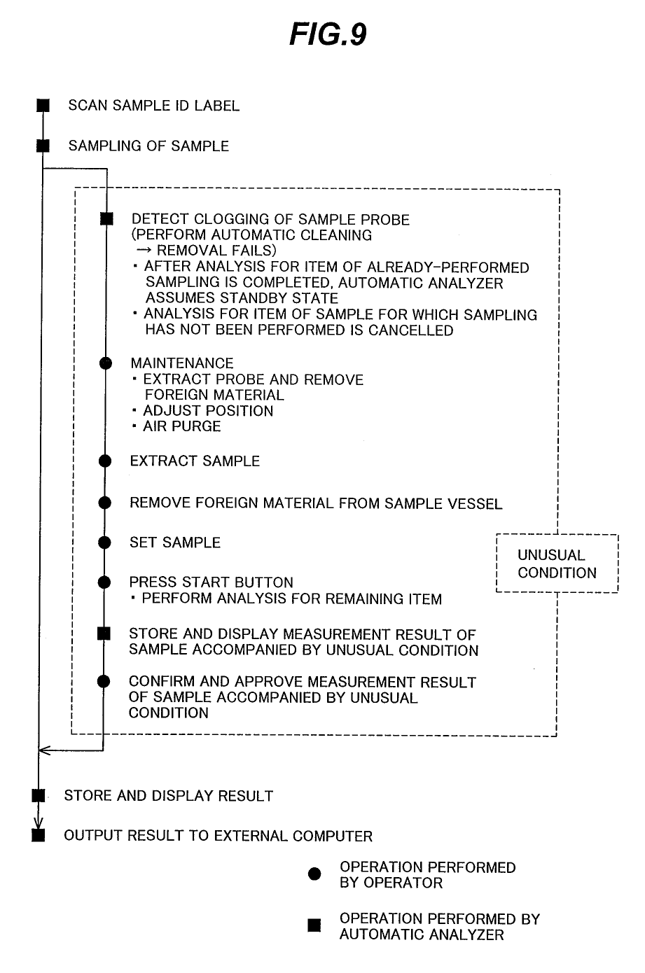

FIG. 9 shows a flow of a process that is to be performed in the case (4). When the sample probe is clogged due to a foreign material such as fibrin during sampling of a sample, the automatic analyzer adds the alarm indicative of detection of clogging of the sample probe to an item of the sample for which sampling has not been performed. In this case, an analysis for an item for a sampled sample continues to be performed. In contrast, an analysis for a remaining item whose sampling has not been performed is cancelled, and the process proceeds to the cleaning operation. When the foreign material cannot be removed from the sample probe by the automatic cleaning operation, the sampling is stopped. The result of the measurement of an item for which the measurement is in progress is output. After that, the automatic analyzer assumes a standby state. In this case, only a sample for which the alarm indicative of detection of clogging of a sample probe is generated is required to be treated. The automatic analyzer is in the state where the analyzer cannot sample a sample. Thus, the automatic analyzer is stopped and recovered.

The sample treated in the case (4) is registered in the measurement result screen 1. The status field for the sample treated in the case (4) indicates “trouble”. The operator selects the sample treated in the case (4) on the measurement result screen 1 and presses the re-measurement button 5. Then, the re-measurement request screen 9 is displayed. The re-measurement request screen 9 shows items (shown in FIG. 9) of operations in a flow format. The items of operations are to be performed by the operator or to be performed by the automatic analyzer. The operator extracts the sample probe in accordance with the guidance and manually removes the foreign material from the sample probe. In addition, the operator performs maintenance such as air purge and adjustment of the position of the probe. After that, the operator resets to the automatic analyzer a sample that is not yet measured and operates the automatic analyzer to cause the automatic analyzer to perform a re-measurement.

The results of the re-measurement are output and stored in the database dedicated for a sample accompanied by an unusual condition. This database is different from the database that is dedicated for a normal sample and designed to store a sample normally treated. The output results are displayed on the measurement result screen 1. The results of the measurement of the samples, which are confirmed and approved by the administrator, are transferred into the database dedicated for a normal sample and output to the external computer in real time or collectively. The automatic analyzer records therein unusual conditions shown in FIG. 9 such as the alarm and the maintenance by using, as a trigger, the detection of the clogging of the sample probe. The unusual conditions are displayed on the special treatment trace screen 10.

In the flow shown in FIG. 9, the performance for searching a sample subjected to the special treatment is improved in a similar way to the aforementioned examples. In addition, reliability of the automatic analyzer is improved by the processes for the confirmation and approval. Furthermore, traceability of the automatic analyzer is ensured by recording the contents of the special treatment for the unusual condition.

### (5) In the Case Where an Alarm Indicative of a Lack of Reagent is Generated

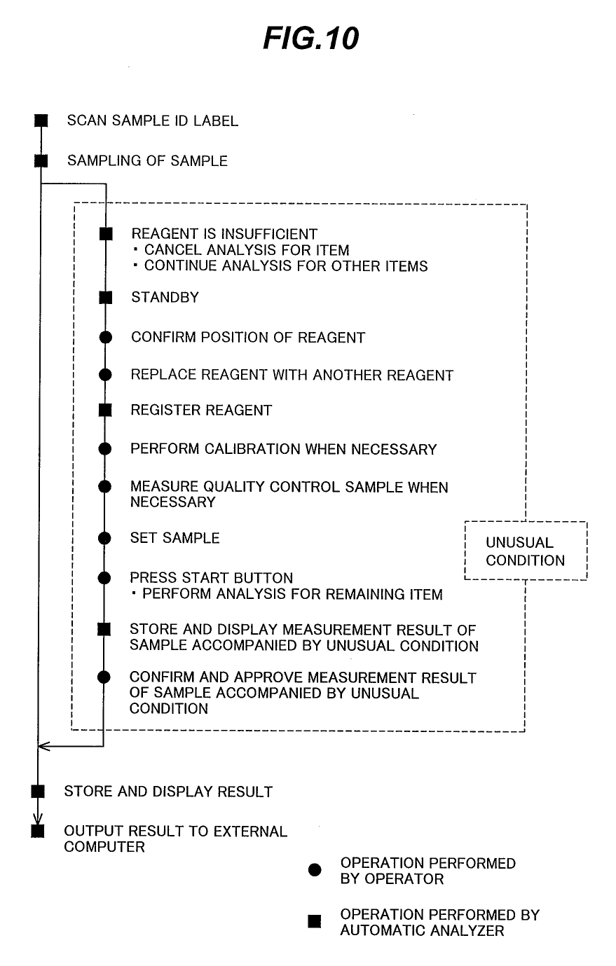

FIG. 10 shows a flow of a process that is to be performed in the case (5). When the remaining amount of the reagent is zero during sampling of a sample, the automatic analyzer adds the alarm indicative of the lack of the reagent to an item indicative of the sampling of the sample for samples that are to be measured next to the sample. An analysis for an item other than the above item continues to be performed. However, an analysis for the item indicative of the sampling of the sample is cancelled. After measurements are performed for all items other than the abovementioned item, the automatic analyzer assumes the standby state. In this case, although only the item for which the amount of the reagent is insufficient is required to be solved, the reagent cannot be replaced during the operation of the automatic analyzer. Thus, the mode of the automatic analyzer is changed from an operation mode to a standby mode, and then the operator recovers the automatic analyzer to the operation mode.

The sample for which the alarm indicative of the lack of the reagent is generated is registered in the measurement result screen 1. The status field for the sample treated in the case (5) indicates “trouble”. The operator selects the sample treated in the case (5) on the measurement result screen 1 and presses the re-measurement button 5. Then, the re-measurement request screen 9 is displayed. The re-measurement request screen 9 shows items (shown in FIG. 10) of operations in a flow format. The items of operations are to be performed by the operator or to be performed by the automatic analyzer. The operator confirms the position of the reagent used for the abovementioned item and replaces a reagent bottle containing the reagent with a new reagent bottle in accordance with the guidance. The automatic analyzer registers the reagent therein and recognizes the position of the new reagent bottle. After that, the automatic analyzer calibrates the position of the new reagent bottle and measures a quality control sample when necessary. Then, the automatic analyzer sets the sample and measures the sample for a remaining item.

The results of the measurement are output and stored in the database dedicated for a sample accompanied by an unusual condition. This database is different from the database that is dedicated for a normal sample and designed to store a sample normally treated. The output measurement results are displayed on the measurement result screen 1. The measurement results of the sample, which are confirmed and approved by the administrator, are transferred into the database dedicated for a normal sample and output to the external computer in real time or collectively. The automatic analyzer records therein unusual conditions shown in FIG. 10 such as the contents of the alarm and the registration of the reagent by using, as a trigger, the generation of the alarm indicative of the lack of the reagent. The unusual conditions are displayed on the special treatment trace screen 10.

In the flow shown in FIG. 10, the performance for searching a sample subjected to the special treatment is improved in a similar way to the abovementioned examples. In addition, reliability of the automatic analyzer is improved by the processes for the confirmation and approval. Furthermore, traceability of the automatic analyzer is ensured by recording the contents of the special treatment for the unusual condition.

The foregoing description discloses that the measurement results of each of the samples subjected to the special treatment for the unusual conditions are displayed on the measurement result screen dedicated for a sample subjected to a special treatment. The samples subjected to the special treatment may be distinguished from normally treated samples by adding identification marks to the samples subjected to the special treatment. In addition, the samples subjected to the special treatment may be distinguished from normally treated samples by adding, to the samples subjected to the special treatment, marks that are different based on the unusual conditions. In the foregoing description, the occurrences of the unusual conditions are determined by the generations of the alarms. Alternatively, the operator may register any sample as a sample accompanied by an unusual condition and register any item as a special item to ensure that the sample is determined to be in an unusual condition.

What is claimed is:

1. An automatic analyzer comprising:
an analyzer for analyzing a sample in accordance with a first analysis item and in response to a first request for a normal treatment; and
a storage mechanism for associating, with identification information used to identify a sample subjected to a special treatment, measurement data on the sample analyzed by the analyzer in accordance with the first analysis item and in response to a second request that is different from the first request and made for the special treatment, and storing the measurement data associated with the identification information.

2. The automatic analyzer according to claim 1,
wherein the first and second requests are made by using measurement numbers different from each other.

3. The automatic analyzer according to claim 1,
wherein the first and second requests are made by using sample identification codes different from each other.

4. The automatic analyzer according to claim 1,
wherein the analysis is performed by the analyzer in response to the second request and in accordance with a second analysis item different from the first analysis item.

5. The automatic analyzer according to claim 1,
wherein the measurement data on the sample analyzed by the analyzer in response to the second request is further associated with a content of the special treatment by the storage mechanism, and the measurement data associated with the content of the special treatment is stored in the storage mechanism.

6. The automatic analyzer according to claim 1, further comprising an output mechanism for outputting only the measurement data on the sample analyzed by the analyzer in response to the second request.

7. An automatic analyzer comprising:
an analyzer for analyzing a sample in accordance with analysis items and in response to a first request for a normal treatment;
a storage mechanism for associating, with identification information used to identify a sample subjected to a special treatment, measurement data on the sample analyzed by the analyzer in accordance with the analysis items and in response to a second request that is different from the first request and made for the special treatment, and storing the measurement data associated with the identification information; and
an output mechanism for outputting the result of an analysis performed for each of the analysis items by using a sample identification code as a unit,
wherein the output mechanism outputs the result of the analysis performed on the sample in accordance with one of the analysis items and in response to the second request for each of the analysis items.

8. An automatic analyzer comprising:
an analyzer for analyzing a sample in accordance with analysis items and in response to a first request for a normal treatment;
a storage mechanism for associating, with identification information used to identify a sample subjected to a special treatment, measurement data on the sample analyzed by the analyzer in accordance with the analysis items and in response to a second request that is different from the first request and made for the special treatment, and storing the measurement data associated with the identification information; and
an output mechanism for outputting the result of an analysis for each of the analysis items by using a patient identification code as a unit,
wherein the output mechanism outputs the result of the analysis performed on the sample in accordance with one of the analysis items and in response to the second request for each of the analysis items.

9. An automatic analyzer comprising:
an analyzer for analyzing a sample in accordance with analysis items and in response to a first request for a normal treatment;
a storage mechanism for associating, with identification information used to identify a sample subjected to a special treatment, measurement data on the sample analyzed by the analyzer in accordance with the analysis items and in response to a second request that is different from the first request and made for the special treatment, and storing the measurement data associated with the identification information; and
an output mechanism for outputting the result of an analysis for each of the analysis items by using a measurement number as a unit,
wherein the output mechanism outputs the result of the analysis performed on the sample in accordance with one of the analysis items and in response to the second request for each of the analysis items.

10. The automatic analyzer according to claim 7,
wherein the result of the analysis performed on the sample subjected to the special treatment and output from the output mechanism includes a content of an unusual condition.

11. The automatic analyzer according to claim 7, further comprising a button for instructing the automatic analyzer to perform a re-analysis for each of the analysis items.

12. The automatic analyzer according to claim 7, further comprising a button for instructing the automatic analyzer to perform at least one of a process for confirming the result of the analysis and a process for approving the result of the analysis.

13. The automatic analyzer according to claim 7,
wherein the output mechanism outputs a method to perform an analysis on a sample accompanied by an unusual condition.

14. The automatic analyzer according to claim 7,
wherein the special treatment is performed for an unusual condition that includes at least one of a lack of a reagent, clogging of a sample probe, a failure to read a barcode, a lack of the sample, a calibration error, a quality control error, and an expiration of the reagent.

15. The automatic analyzer according to claim 7, further comprising a button for registering a sample subjected to the special treatment by an operator.
